(19) United States
(12) Patent Application Publication
(10) Pub. No.: US 2020/0315965 A1
(43) Pub. Date: Oct. 8, 2020

(54) NANOEMULSION CONCENTRATE FORMULATIONS AND METHODS

(71) Applicant: CANNASOL TECHNOLOGIES, LLC, Berea, OH (US)

(72) Inventor: Joshua Michael Detzel, Richfield, OH (US)

(21) Appl. No.: 16/838,471

(22) Filed: Apr. 2, 2020

Related U.S. Application Data
(60) Provisional application No. 62/828,078, filed on Apr. 2, 2019.

(51) Int. Cl. A61K 9/107; A61K 47/14; A61K 47/24; A61K 47/26; A61K 47/46; A23L 33/12; A23L 33/105; A23L 33/125

(52) U.S. Cl. CPC A61K 9/1075; A61K 47/14; A61K 47/24; A61K 47/26; A61K 31/05; A23L 33/12; A23L 33/105; A23L 33/125; A61K 47/46

(57) ABSTRACT

The disclosure provides a stable nanoemulsion concentrate suitable for formulation of one or more non-polar active ingredients, for example a cannabinoid, together with methods of making and using the same.

### CROSS REFERENCE TO RELATED APPLICATION

This patent application claims priority to, and the benefit of, U.S. Provisional Application No. 62/828,078, filed on Apr. 2, 2019, the contents of which are hereby incorporated by reference in its entirety.

### FIELD

The present invention relates to nanoemulsions which contain non-polar active ingredients, intended for use in foods, beverages, supplements, or pharmaceutical products. More specifically, the invention relates to a stable nanoemulsion concentrate, comprised of relatively low amounts of natural and naturally-derived surfactants, consumer-friendly non-polar solvents and consumer-friendly aqueous phase ingredients, with an average dispersed-phase particle diameter below 100 nanometers. This nanoemulsion concentrate affords enhanced bioavailability of the non-polar active ingredients and a high degree of optical transparency upon dilution into an otherwise-transparent consumable product.

### BACKGROUND

Many biologically active food, beverage, supplement and drug ingredients are poorly soluble in water, making formulation of aqueous food, beverage, supplement and pharmaceutical products difficult. Furthermore, these poorly water-soluble active ingredients frequently have low oral bioavailability and are inconsistently absorbed through the body's mucous membranes and from the digestive tract into the blood stream.

Cannabinoids, such as cannabidiol (CBD) and tetrahydrocannabinol (THC), are non-polar compounds and suffer from both of the problems mentioned above; CBD and THC have aqueous solubilities of approximately 0.013 and 0.003 milligrams per milliliter, respectively. Clinical studies have reported the oral bioavailability of CBD and THC as low as approximately 5-10%, and absorption has been reported to fluctuate greatly depending on various factors, such as the fat content of meals consumed by the study participants prior-to dosing and other factors.

For cannabinoids and similarly insoluble non-polar active ingredients, some of these formulation and bioavailability problems can be partially overcome by processing these ingredients into oil-in-water nanoemulsion concentrates, with oil-phase (dispersed-phase) average particle diameters under about 300 nanometer (nm). This process frequently involves blending the non-polar active ingredient(s) with one or more non-polar solvents, surfactants, water, and other ingredients, then processing the mixture with an ultrasonic homogenizer, high-pressure homogenizer, or a high-shear mixer to yield an emulsion with average oil-phase droplet diameter less than about 300 nm, dispersed in an aqueous continuous phase. However, many of the cannabinoid nanoemulsion formulations currently available do not have an average particle size below 100 nm, and frequently utilize consumer-unfriendly synthetic surfactants.

There is a need for improved formulations of poorly water-soluble active ingredients, such as CBD and THC.

### SUMMARY

The present disclosure provides a nanoemulsion concentrate comprised of a primary surfactant which is derived from natural sources, natural secondary surfactants, natural and/or consumer-friendly synthetic non-polar solvents, optional antioxidants/stabilizers, and one or more of non-polar cannabinoids, specifically cannabidiol (CBD) and tetrahydrocannabinol (THC), or other non-polar active ingredients requiring an emulsifier blend with an HLB of about 13 or higher. This nanoemulsion concentrate may be produced using ultrasonic homogenization or high-pressure homogenization. This nanoemulsion concentrate is a stable oil-in-water nanoemulsion with an average oil-phase particle diameter below 100 nm. This nanoemulsion concentrate has low food, beverage or pharmaceutical taste contribution, and, in preferred embodiments, a total surfactant content that does not substantially exceed the weight percentage of the oil-phase components, which comprise non-polar solvents, non-polar active ingredients and other non-polar ingredients.

The disclosure further provides an oil/surfactant pre-mixture, which when combined with an aqueous medium, will provide a nanoemulsion, e.g., as described above.

The disclosure further provides methods of making and using these formulations.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

FIG. 1 illustrates the internal structure of the nanoemulsion concentrate by providing a cross section of the oil-phase and surrounding aqueous phase.

FIG. 2 illustrates a processing scheme for producing the nanoemulsion concentrate.

### DETAILED DESCRIPTION

For oil-in-water nanoemulsions, there is an inverse correlation between average oil-phase particle size and: oral bioavailability of the non-polar active ingredient, absorption of the non-polar active ingredient through the body's mucous membranes, optical transparency of the emulsion, stability of the emulsion against separation, and the degradation rate of light-sensitive ingredients contained within the oil-phase. Absorption of a non-polar active ingredient in the small intestine can be further enhanced by the presence of certain non-polar solvents, such as long-chain triglyceride oils. In certain embodiments, the nanoemulsion of the disclosure comprising one or more non-polar active ingredients has an oil-phase particle-size distribution where the vast majority of oil-phase particles have diameters less than 100 nm, and an average oil-phase droplet diameter around about 50 nm, or smaller.

Beyond particle size, there are several other important considerations when formulating a non-polar active ingredient into a nanoemulsion for food, beverage, or pharmaceutical applications, such as the taste contribution of the surfactants and non-polar solvents, the necessity to minimize the amount of surfactant used, and the avoidance of harsh or consumer-unfriendly synthetic surfactants. Ideally, the surfactant(s) should impart little to no taste into the food, beverage, supplement, or pharmaceutical product. Also, ideally, the surfactant content of the nanoemulsion should be sufficiently low that the surfactant(s) does not substantially contribute to undesirable foaming of the consumer product, alter the mouth-feel of the consumer product, or irritate the digestive tract of persons or animals who ingest the consumer product. In certain embodiments, the surfactants used in the nanoemulsions of the disclosure are, by weight percentage, less than or equal to the weight percentage of the non-polar oil-phase components.

Consumers increasingly avoid products that contain synthetic surfactants like polysorbates, sorbitan esters, polyethylene glycol, sodium lauryl sulfate and others due to both real and perceived health risks. In certain embodiments, the surfactants present in the nanoemulsion of the disclosure are natural, naturally-derived, or derived from natural sources.

Surfactants have a property referred to as hydrophilic-lipophilic balance (HLB), which is a measure of how hydrophilic or lipophilic a given surfactant, or surfactant blend is. The HLB of surfactants generally ranges from 0 to 20, with 0 being the most lipophilic and 20 being the most hydrophilic. Surfactants used to produce oil-in-water emulsions often fall within the range of about 8 to 16 on the HLB scale. In certain nanoemulsions of the disclosure containing non-polar cannabinoids, such as CBD, THC and other similar non-polar active ingredients, the surfactant or blend of surfactants in the nanoemulsion have a relatively high overall HLB, e.g., an HLB of about 13 or higher.

It is possible to achieve an ideal oil-phase particle size using surfactant blends consisting of certain high-HLB synthetic surfactants, such as polysorbates, combined with low-HLB surfactants such as sorbitan esters, at relatively high concentrations; however, it is quite difficult to achieve this ideal particle size using only surfactants that are natural, naturally-derived, or that are derived from natural sources, even when the surfactant mixture has the requisite overall HLB, especially at surfactant concentrations that do not exceed the concentration of the oil-phase components. Furthermore, both natural and synthetic surfactants often impart undesirable taste into the nanoemulsion concentrate. For example, both polysorbates (consumer-unfriendly, synthetic) and saponins (consumer-friendly, natural) can contribute undesirable taste to a finished product if utilized at high concentrations. This consideration is especially important when producing an emulsion that contains an active ingredient which already has negative taste qualities, such as CBD, which is naturally quite bitter.

Sucrose fatty acid esters are a class of surfactants which are considered to be either natural or derived from natural sources, therefore garnering high consumer acceptance. Sucrose fatty acid esters are unique due to their capacity to cover a wide HLB spectrum. Blends of sucrose esters, such as sucrose palmitates and sucrose stearates, which contain high concentrations of sucrose monoesters have a relatively high HLB that ranges to 16. However, sucrose fatty acid esters can be problematic for formulation due to their high viscosity in solution. Furthermore, though sucrose fatty acid esters are among the best natural emulsifiers, it is difficult, if not impossible to achieve an average oil-phase particle size substantially smaller than 50-100 nm using sucrose fatty acid esters, alone, as a surfactant, especially for formulations containing high concentrations of cannabinoids.

Therefore, in certain embodiments, the disclosure provides nanoemulsion concentrates with average oil-phase particle diameter around about 50 nm, or smaller, comprised of surfactants that are natural, naturally derived, or derived from natural sources, and consumer-friendly non-polar solvents which is needed to allow for the production of consumer-friendly, stable, transparent, good-tasting, food, beverage and orally-administered supplement and pharmaceutical products which contain poorly water-soluble and otherwise poorly bioavailable non-polar active ingredients.

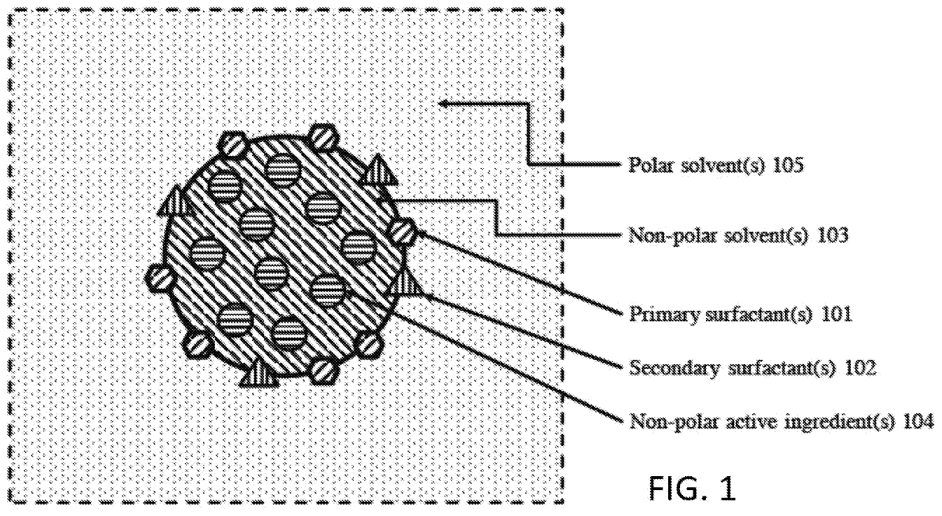

FIG. 1 illustrates the internal structure of the nanoemulsion concentrate by providing a cross section of an oil-phase particle and the surrounding aqueous phase. In preferred embodiments, the nanoemulsion concentrate is comprised of the following ingredients: a) a primary surfactant 101 comprising one sucrose fatty acid ester or mixture of sucrose fatty acid esters selected from among sucrose palmitates, sucrose stearates, or other food-safe sucrose fatty acid esters, ideally with an HLB of 14 or greater, b) one or more secondary surfactant(s) 102 selected from among phospholipid-containing ingredients, including, but not limited to, liquid lecithins, de-oiled lecithins, or modified lecithins, and saponins, including, but not limited to those derived from Quillaja saponaria, c) one or more non-polar solvent(s) 103 selected from among mixtures of medium chain triglycerides, mixtures of long chain triglycerides, coconut oil, fractionated coconut oil, olive oil, corn oil, sunflower oil, fish oil and other non-polar oils commonly used in food or beverage products, e) a non-polar active ingredient(s) 104 selected from among any one or more of cannabidiol, tetrahydrocannabinol, another non-polar cannabinoid compound or another non-polar active ingredient, and optionally, d) one or more polar solvent(s) 105, comprising an aqueous continuous phase, selected from among water and various non-water polar compounds, such as glycerin, propylene glycol, or other non-water polar compounds commonly used in food or beverage products.

In some embodiments, the sucrose fatty acid esters are present in an amount between about 1% and 15%, by weight of the concentrate. In some embodiments, the sucrose fatty acid esters are present in an amount of from 2.5% to 12.5%, e.g., from 4% to 10%, from 4% to 8%, or from 5% to 7.5% by weight of the concentrate. In some embodiments, the primary surfactant 101 is a mixture of sucrose palmitate(s) and sucrose stearate(s), optionally wherein the mixture of sucrose palmitate(s) and sucrose stearate(s) has an overall monoester content of about 75% and a diester content of about 20% and has an HLB of about 16.

In some embodiments, the secondary surfactant 102 is present in an amount between about 0.75% and 7%, by weight, of the concentrate. In some embodiments, the secondary surfactant 102 is present in an amount of from 1% to 5%, e.g., from 1.5% to 5%, from 1.5% to 4.5%, from 1.5% to 4%, from 1.5% to 3.7%, from 1.5% to 3.5%, from 1.5% to 3%, from 1.5% to 2.5% or from 1.9% to 3.7% by weight of the concentrate. In some embodiments, the secondary surfactant 102 are selected from lecithins, saponins, e.g., saponins derived from Quillaja saponaria, and mixtures thereof. The lecithins may be selected from liquid lecithins, de-oiled lecithins, and modified lecithins. In preferred embodiments, the secondary surfactant 102 comprises lecithins, e.g, selected from liquid lecithins, de-oiled lecithins, or modified lecithins, and saponins, e.g., saponins derived from Quillaja saponaria. In some embodiments, the lecithins are present in an amount of from 0.5% to 4%, e.g., from 0.5% to 3.5%, from 1% to 3.5%, from 1% to 3%, from 1% to 2.6%, from 1% to 2.5%, or from 1.1% to 2.5% by weight of the concentrate and the saponins are present in an amount of from 0.3% to 2%, e.g., from 0.5% to 1.6%, from 0.5% to 1.5%, from 0.6% to 1.3%, from 0.6% to 1.2%, from 0.7% to 1.3%, or from 0.7% to 1.2%, by weight of the concentrate. In certain embodiments, the lecithins are present in an amount of from 1% to 2.5% by weight of the concentrate and the saponins are present in an amount of from 0.7% to 1.2% by weight of the concentrate.

In some embodiments, the non-polar solvent(s) 103 is present in an amount between about 2.5% and 12.5%, by weight, of the concentrate. In some embodiments, the non-polar solvent(s) 103 is present in an amount of from 3% to 12%, e.g., from 4% to 10%, from 5% to 10%, from 4% to 8%, from 5% to 8%, or from 5% to 7.5% by weight of the concentrate. In some embodiments, the non-polar solvent(s) 103 are selected from long-chain triglyceride-containing non-polar solvents, medium-chain triglyceride-containing solvents, and mixtures thereof. In some embodiments, the non-polar solvent(s) 103 comprises a long-chain triglyceride-containing non-polar solvent, e.g., olive oil. In some embodiments, the non-polar solvent(s) 103 comprises a medium-chain triglyceride-containing non-polar solvent, e.g., coconut oil. In some embodiments, the non-polar solvent(s) 103 comprises a combination of a long-chain triglyceride-containing non-polar solvents and a medium-chain triglyceride-containing solvents, e.g., a combination of olive oil and coconut oil.

In some embodiments, the non-polar active ingredient(s) 104 is present in an amount between 2.5% and 10%, by weight, of the concentrate. In some embodiments, the non-polar active ingredient(s) 104 is present in an amount of from 4% to 8%, from 4.5% to 8%, or from 5% to 7.5% by weight of the concentrate. In preferred embodiments, the non-polar active ingredient(s) 104 is cannabidiol. In other embodiments, the non-polar active ingredient(s) 104 is tetrahydrocannabinol. In other embodiments, the non-polar active ingredient(s) 104 is hemp extract.

In some embodiments, the polar solvent(s) 105 is present in an amount between about 58% and 92%, by weight, of the concentrate. In some embodiments, the polar solvent(s) 105 is present in an amount of from 60% to 85%, from 65% to 85%, from 70% to 85%, from 70% to 80%, from 75% to 85%, or from 80% to 85% by weight of the concentrate. In some embodiments, the polar solvent(s) 105 comprises a combination of water and glycerin.

In some embodiments, this nanoemulsion concentrate has low food, beverage or pharmaceutical taste contribution, and a total surfactant content that does not substantially exceed the weight percentage of the oil-phase components, which comprise non-polar solvents, non-polar active ingredients and other non-polar ingredients.

For certain applications, the inclusion of one or more non-polar antioxidants may be beneficial to slow or halt the degradation of the non-polar active ingredient(s). These oil-soluble antioxidants are ideally selected from among mixed tocopherols, quercetin, curcumin or other suitable oil-soluble antioxidants, with the ability to slow or halt the degradation of the non-polar active ingredient(s). For certain applications, the addition of anti-bacterial and/or anti-fungal agents may be beneficial. These anti-bacterial and/or anti-fungal agents are ideally non-polar, natural anti-bacterial and/or anti-fungal agents selected from among thymol, carvacrol, lauric acid, or other naturally occurring anti-bacterial and/or anti-fungal agents.

In some embodiments, the nanoemulsion concentrate comprises, by weight: a) about 4% to 8% sucrose fatty acid esters, e.g., a mixture of sucrose palmitate(s) and sucrose stearate(s), b) about 0.6% to 1.3% saponins, e.g., saponins derived from Quillaja saponaria, and about 1% to 2.6% lecithins e.g., de-oiled soy lecithin as secondary surfactants, c) about 4% to 8% non-polar solvent(s), e.g., olive oil, d) about 70% to 85% polar solvent(s), e.g., a combination of water and glycerin, and e) about 4% to 8% non-polar active ingredient(s), e.g., cannabidiol.

In some embodiments, the nanoemulsion concentrate comprises, by weight: a) about 5% to 7.5% sucrose fatty acid esters, e.g., a mixture of sucrose palmitate(s) and sucrose stearate(s), b) about 0.7% to 1.2% saponins, e.g., saponins derived from Quillaja saponaria, and about 1.1% to 2.5% lecithins e.g., de-oiled soy lecithin as secondary surfactants, c) about 5% to 7.5% non-polar solvent(s), e.g., olive oil, d) about 70% to 85% polar solvent(s), e.g., a combination of water and glycerin, and e) about 5% to 7.5% non-polar active ingredient(s), e.g., cannabidiol.

In some embodiments, the nanoemulsion concentrate comprises, by weight: a) about 4.5% to 6% sucrose fatty acid esters, e.g., a mixture of sucrose palmitate(s) and sucrose stearate(s), b) about 0.6% to 1% saponins, e.g., saponins derived from Quillaja saponaria, and about 1% to 1.8%% lecithins e.g., de-oiled soy lecithin as secondary surfactants, c) about 4% to 6% non-polar solvent(s), e.g., olive oil, d) about 80% to 85% polar solvent(s), e.g., a combination of water and glycerin, and e) about 4.5% to 5.5% non-polar active ingredient(s), e.g., cannabidiol.

In some embodiments, the nanoemulsion concentrate comprises, by weight: a) about 4.5% to 5.5% sucrose fatty acid esters, e.g., a mixture of sucrose palmitate(s) and sucrose stearate(s), b) about 0.7% to 0.8% saponins, e.g., saponins derived from Quillaja saponaria, and about 1.5% to 1.8%% lecithins e.g., de-oiled soy lecithin as secondary surfactants, c) about 4% to 6% non-polar solvent(s), e.g., olive oil, d) about 80% to 85% polar solvent(s), e.g., a combination of water and glycerin, and e) about 4.5% to 5.5% non-polar active ingredient(s), e.g., cannabidiol.

In some embodiments, the nanoemulsion concentrate comprises, by weight: a) about 5% to 6% sucrose fatty acid esters, e.g., a mixture of sucrose palmitate(s) and sucrose stearate(s), b) about 0.7% to 1% saponins, e.g., saponins derived from Quillaja saponaria, and about 1% to 1.3% lecithins e.g., de-oiled soy lecithin as secondary surfactants, c) about 4% to 6% non-polar solvent(s), e.g., olive oil, d) about 80% to 85% polar solvent(s), e.g., a combination of water and glycerin, and e) 4.5%-5.5% non-polar active ingredient(s), e.g., cannabidiol.

In some embodiments, the nanoemulsion concentrate comprises, by weight: a) about 7% to 8% sucrose fatty acid esters, e.g., a mixture of sucrose palmitate(s) and sucrose stearate(s), b) about 1% to 1.2% saponins, e.g., saponins derived from Quillaja saponaria, and about 2.3% to 2.7% lecithins e.g., de-oiled soy lecithin as secondary surfactants, c) about 7% to 8% non-polar solvent(s), e.g., olive oil, d) about 70% to 75% polar solvent(s), e.g., a combination of water and glycerin, and e) about 7% to 8% non-polar active ingredient(s), e.g., cannabidiol.

In certain embodiments, the nanoemulsion concentrate comprises, by weight, about: a) about 5% sucrose fatty acid esters, specifically a mixture of sucrose palmitate(s) and sucrose stearate(s) with an HLB of about 16, as the primary surfactant, b) about 0.8% saponins, derived from Quillaja saponaria, and about 1.7% de-oiled soy lecithin as secondary surfactants, c) about 5% olive oil as a non-polar solvent, d) about 79% water and about 3% glycerin as the aqueous phase, and e) about 5% cannabidiol as the non-polar active ingredient.

In certain embodiments, the nanoemulsion concentrate comprises, by weight, about: a) about 5% sucrose fatty acid esters, specifically a mixture of sucrose palmitate(s) and sucrose stearate(s) with an HLB of about 16, as the primary surfactant, b) about 0.8% saponins, derived from Quillaja saponaria, and about 1.7% de-oiled soy lecithin as secondary surfactants, c) about 5% olive oil as a non-polar solvent, d) about 79% water and about 3% glycerin as the aqueous phase, and e) about 5% hemp seed as the non-polar active ingredient.

In certain embodiments, the nanoemulsion concentrate comprises, by weight, about: a) about 5.5% sucrose fatty acid esters, specifically a blend of sucrose palmitate(s) and sucrose stearate(s) with an HLB of about 16, as the primary surfactant, b) about 0.9% saponins, derived from Quillaja saponaria, and about 1.1% de-oiled soy lecithin as secondary surfactants, c) about 5% olive oil as a non-polar solvent, d) about 79% water and about 3% glycerin as the aqueous phase, and e) about 5% cannabidiol as the non-polar active ingredient.

In certain embodiments, the nanoemulsion concentrate comprises, by weight, about: a) about 7.5% sucrose fatty acid esters, specifically a mixture of sucrose palmitate(s) and sucrose stearate(s) with an HLB of about 16, as the primary surfactant, b) about 1.1% saponins, derived from Quillaja saponaria, and about 2.5% de-oiled soy lecithin as secondary surfactants, c) about 7.5% olive oil as a non-polar solvent, d) about 71% water and about 3% glycerin as the aqueous phase, and e) about 5% cannabidiol as the non-polar active ingredient.

In some embodiments, the nanoemulsion concentrate comprises, by weight, about: a) 5% sucrose fatty acid esters, specifically a blend of sucrose palmitate(s) and sucrose stearate(s) with an HLB of about 16, as the primary surfactant, b) 0.7% saponins, derived from Quillaja saponaria, and 1.7% de-oiled soy lecithin as secondary surfactants, c) 5% olive oil as a long-chain triglyceride-containing non-polar solvent, and 0.5% coconut oil as a source of medium-chain triglycerides, including lauric acid, d) 78.8% water and 3.3% glycerin as the aqueous phase, and e) 5% cannabidiol as the non-polar active ingredient.

In other embodiments, the nanoemulsion concentrate comprises, by weight, about: a) 7.5% sucrose fatty acid esters, specifically a blend of sucrose palmitate(s) and sucrose stearate(s) with an HLB of about 16, as the primary surfactant, b) 1.1% saponins, derived from Quillaja saponaria, and 2.5% de-oiled soy lecithin as secondary surfactants, c) 7.5% olive oil as a long chain triglyceride-containing non-polar solvent, d) 68.9% water and 5% glycerin as the aqueous phase, e) 7.5% cannabidiol as the non-polar active ingredient.

In other embodiments, the nanoemulsion concentrate comprises, by weight, about: a) 5% sucrose fatty acid esters, specifically a blend of sucrose palmitate(s) and sucrose stearate(s) with an HLB of about 16, as the primary surfactant, b) 0.7% saponins, derived from Quillaja saponaria, and 1.7% de-oiled soy lecithin as secondary surfactants, c) 5% olive oil as a long chain triglyceride-containing non-polar solvent, 1.3% fractionated coconut oil as a medium chain triglyceride-containing non-polar solvent, d) 76.3% water and 5% glycerin as the aqueous phase, e) 5% cannabidiol as the non-polar active ingredient.

In other embodiments, the nanoemulsion concentrate comprises, by weight, about: a) 5% sucrose fatty acid esters, specifically a blend of sucrose palmitate(s) and sucrose stearate(s) with an HLB of about 16, as the primary surfactant, b) 0.7% saponins, derived from Quillaja saponaria, and 1.7% de-oiled soy lecithin as secondary surfactants, c) 5% olive oil as a long chain triglyceride-containing non-polar solvent, 1.3% fractionated coconut oil as a medium chain triglyceride-containing non-polar solvent, d) 76.3% water and 5% glycerin as the aqueous phase, e) 5% cannabidiol as the non-polar active ingredient, f) 0.5% mixed tocopherols as an oil-soluble antioxidant.

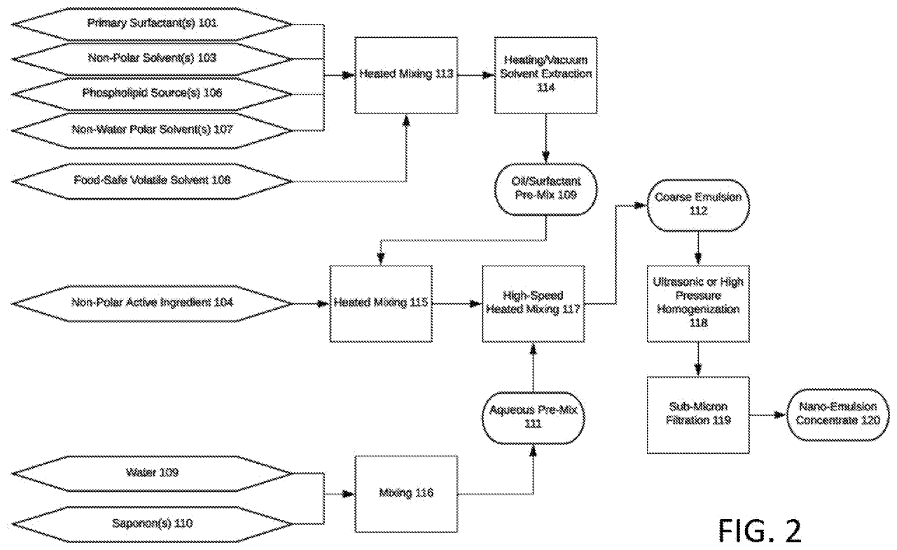

FIG. 2 illustrates a method of producing the nanoemulsion concentrate, which is as follows: the primary surfactant 101, the non-water polar solvents 107 (if present), the non-polar solvent(s) 103 and the phospholipid source(s) 106 (if present), undergo heated mixing 113. Ethanol, or another food-safe volatile solvent 108 with water content of less than about 25%, is added during the heated mixing step 113 for the purpose of enhancing solvation and decreasing mixing time. Once the mixture is visually determined to be homogeneous, a combination of heating and vacuum solvent extraction 114 may be used to remove substantially all or most of the food-safe volatile solvent(s), resulting in the formation of the surfactant/oil pre-mix 109. After the surfactant/oil pre-mixture is formed, the non-polar active ingredient(s) 104 is then mixed with the surfactant/oil pre-mixture, under heated mixing 115, until the non-polar active ingredient(s) 104 is thoroughly distributed and/or dissolved in the resulting mixture. The water 109 and the saponin(s) 110 (if present in the formulation) undergo mixing 116 until the saponin(s) 110 fully dissolve into the water 109 to form the aqueous pre-mix 111. The aqueous pre-mix is heated to 40-50 degrees Celsius and added, under high-speed heated mixing 117, to the heated surfactant/oil pre-mix, resulting in the formation of a coarse emulsion 112 with average oil phase particle size above 300 nm. This coarse emulsion 112 is then subjected to ultrasonic or high pressure homogenization 118 to reduce oil-phase droplet diameter to the desired level. Finally, the processed emulsion is filtered to a sub-micron level 119 to remove particulates, yielding the final nanoemulsion concentrate 120.

Ideal mixing temperature for heated mixing steps 113, 115 and 117 ranges from 60 to 80 degrees Celsius. Ideal ultrasonic processing 118 conditions for converting the coarse emulsion 112 to the nanoemulsion concentrate 120 range from: an ultrasonic probe amplitude of 75-90 microns, most preferably 80 microns, an emulsion temperature maintained between 45-75 degrees Celsius, most preferably 65 degrees Celsius, and a processing rate of 2-10 liters of emulsion per hour per square inch of the ultrasonic horn's tip area. This nanoemulsion concentrate, when diluted into a food, supplement, beverage, or orally-administered pharmaceutical product, has relatively low to imperceptible taste, a high degree of optical transparency, and improves the bioavailability of the cannabinoid or other non-polar active ingredient(s).

The disclosure thus provides a liquid nanoemulsion concentrate (Formulation 1), comprising:
a. a primary surfactant comprising a sucrose fatty acid ester or a mixture of sucrose fatty acid esters, wherein the total amount of sucrose fatty acid esters is between about 1% and about 15%, by weight of the concentrate;
b. one or more secondary surfactants selected from among lecithins, e.g., de-oiled lecithin, saponins, e.g., saponin derived from Quillaja saponaria, and mixtures thereof, wherein the total amount of secondary surfactants is between about 0.3% and about 10%, by weight of the concentrate;
c. one or more non-polar solvents, in a total amount between about 1% and about 18%, by weight, of the concentrate;
d. a non-polar active ingredient in a total amount between about 1% and about 15%, by weight, of the concentrate; and
e. an aqueous continuous phase consisting of one or more polar solvents in a total amount between about 45% and about 96%, by weight of the concentrate.

For example, in particular embodiments the disclosure provides:
1.1. Formulation 1, wherein the sucrose fatty acid ester or a mixture of sucrose fatty acid esters is selected from the group consisting of sucrose palmitates, sucrose stearates, and mixtures thereof.
1.2. Formulation 1 or 1.1 wherein the sucrose fatty acid ester or mixture of sucrose fatty acid esters has an HLB of about 14 to 16.
1.3. Any previous formulation, wherein the total amount of sucrose fatty acid esters is from 2.5% to 12.5%, e.g., from 4% to 10%, from 4% to 8%, or from 5% to 7.5% by weight of the concentrate.
1.4. Any previous formulation, wherein the primary surfactant is a mixture of sucrose palmitate(s) and sucrose stearate(s), optionally wherein the mixture of sucrose palmitate(s) and sucrose stearate(s) has an overall monoester content of about 75% and a diester content of about 20% and has an HLB of about 16.
1.5. Any previous formulation, wherein the sucrose fatty acid ester or a mixture of sucrose fatty acid esters has an HLB of about 14 to 16.
1.6. Any previous formulation, wherein the secondary surfactant is present in an amount of from 1% to 5%, e.g., from 1.5% to 5%, from 1.5% to 4.5%, from 1.5% to 4%, from 1.5% to 3.7%, from 1.5% to 3.5%, from 1.5% to 3%, from 1.5% to 2.5% or from 1.9% to 3.7% by weight of the concentrate.
1.7. Any previous formulation, wherein the secondary surfactant comprises liquid lecithins, de-oiled lecithins, modified lecithins, or mixtures thereof
1.8. Any previous formulation wherein the secondary surfactant comprises a saponin derived from Quillaja saponaria.
1.9. Any previous formulation, wherein the secondary surfactants comprise a mixture of lecithins and saponins.
1.10. Any previous formulation, wherein the lecithins are present in an amount of from 0.5% to 4%, e.g., from 0.5% to 3.5%, from 1% to 3.5%, from 1% to 3%, from 1% to 2.6%, from 1% to 2.5%, or from 1.1% to 2.5% by weight of the concentrate and the saponins are present in an amount of from 0.3% to 2%, e.g., from 0.5% to 1.6%, from 0.5% to 1.5%, from 0.6% to 1.3%, from 0.6% to 1.2%, from 0.7% to 1.3%, or from 0.7% to 1.2%, by weight of the concentrate.
1.11. Any previous formulation, wherein the lecithins are present in an amount of from 1% to 2.6% by weight of the concentrate and the saponins are present in an amount of from 0.6% to 1.3% by weight of the concentrate.
1.12. Any previous formulation, wherein the lecithins are present in an amount of from 1.1% to 2.5% by weight of the concentrate and the saponins are present in an amount of from 0.7% to 1.2% by weight of the concentrate.
1.13. Any previous formulation, wherein the secondary surfactants comprise a mixture of a de-oiled lecithin and one or more saponins.
1.14. Any previous formulation, wherein the one or more nonpolar solvents comprise triglycerides.
1.15. Any previous formulation, wherein the one or more nonpolar solvents are selected from medium-chain triglycerides, long-chain triglycerides, coconut oil, fractionated coconut oil, olive oil, corn oil, sunflower oil, fish oil, and mixtures thereof
1.16. Any previous formulation, wherein the one or more nonpolar solvents comprise a long-chain triglyceride-containing non-polar solvent, e.g., olive oil.
1.17. Any previous formulation, wherein the one or more nonpolar solvents comprise a medium-chain triglyceride-containing non-polar solvent, e.g., coconut oil.
1.18. Any previous formulation, wherein the one or more nonpolar solvents comprise a combination of a long-chain triglyceride-containing non-polar solvents and a medium-chain triglyceride-containing solvents, e.g., a combination of olive oil and coconut oil.
1.19. Any previous formulation, wherein the one or more nonpolar solvents are present in an amount of from 3% to 12%, e.g., from 4% to 10%, from 5% to 10%, from 4% to 8%, from 5% to 8%, or from 5% to 7.5% by weight of the concentrate.
1.20. Any previous formulation wherein the non-polar active ingredient is selected from non-polar cannabinoid compounds.
1.21. Any previous formulation wherein the non-polar active ingredient is selected from cannabidiol, tetrahydrocannabinol, hemp or cannabis plant extracts containing greater than 50% total cannabinoids, and mixtures thereof
1.22. Any previous formulation wherein the non-polar active ingredient is cannabidiol.
1.23. Any previous formulation, wherein the non-polar active ingredient is tetrahydrocannabinol.
1.24. Any previous formulation, wherein the non-polar active ingredient is hemp oil.
1.25. Any previous formulation, wherein the non-polar active ingredient is present in an amount between 2.5% and 10%, e.g., from 4% to 8%, from 4.5% to 8%, or from 5% to 7.5%, by weight of the concentrate.
1.26. Any previous formulation wherein the non-polar active ingredient is cannabidiol, wherein the cannabidiol is substantially free of other cannabinoids, e.g., at least 99% free of other cannabinoids.
1.27. Any previous formulation wherein the non-polar active ingredient is cannabidiol, wherein the cannabidiol is substantially free of other cannabinoids, e.g., at least 99% free of other cannabinoids, and is isolated from hemp.
1.28. Any previous formulation, wherein the polar solvent is present in an amount between about 58% and 92%, e.g., from 60% to 85%, from 65% to 85%, from 70% to 85%, from 70% to 80%, from 75% to 85%, or from 80% to 85%, by weight of the concentrate.
1.29. Any previous formulation, wherein the polar solvent is selected from water, glycerin, propylene glycol, and mixtures thereof
1.30. Any previous formulation, wherein the polar solvent comprises a mixture of water and glycerin.
1.31. Any previous formulation wherein the polar solvent is a beverage.
1.32. Any previous formulation wherein the total surfactants in the nanoemulsion have an overall HLB of about 13 or higher.
1.33. Any previous formulation wherein the average oil-phase particle diameter is less than 100 nm.
1.34. Any previous formulation wherein the average oil-phase particle diameter is less than 50 nm.
1.35. Any previous formulation wherein the average oil-phase particle diameter is about 40-60 nm.
1.36. Any previous formulation wherein the oil-phase particles have a 90^{th} percentile volume-weighted particle size (Dv90) of less than 60 nm.
1.37. Any previous formulation wherein the oil-phase particles have a Dv90 of 30-60 nm.
1.38. Any previous formulation wherein the percentage of oil-phase particles which have a diameter of under 100 nm is at least 95%.
1.39. Any previous formulation wherein the percentage of oil-phase particles which have a diameter of under 100 nm is at least 98%.
1.40. Any previous formulation wherein the percentage of oil-phase particles which have a diameter of under 100 nm is at least 99%.
1.41. Any previous formulation further comprising an oil-soluble antioxidant.
1.42. Any previous formulation further comprising mixed tocopherols as an oil-soluble antioxidant.
1.43. Any previous formulation further comprising about 0.5% mixed tocopherols as an oil-soluble antioxidant.
1.44. Any previous formulation wherein the formulation comprises, by weight of the formulation, about 5% to 7.5% sucrose fatty acid esters, about 2.4% to 3.6% secondary surfactants, about 5% to 7.5% non-polar solvents, about 5% to 7.5% non-polar active ingredient, and about 73.9% to 82.6% polar solvents.
1.45. Any previous formulation wherein the formulation comprises, by weight of the formulation, about 5% to 7.5% sucrose fatty acid esters, about 2.4% to 3.6% secondary surfactants, about 6.3% to 9.5% non-polar solvents, about 5% to 7.5% non-polar active ingredient, and about 71.9% to 81.3% polar solvents.
1.46. Any previous formulation wherein the formulation comprises between about 4.5% and about 12.5%, by weight of the formulation, of sucrose fatty acid ester or mixture thereof.
1.47. Any previous formulation, wherein the surfactants are natural or derived from natural sources.
1.48. Any previous formulation, wherein the weight percentage of the surfactants does not substantially exceed that of the non-polar components.
1.49. Any previous formulation, wherein the nanoemulsion concentrate comprises, by weight: a) about 4% to 8% sucrose fatty acid esters, e.g., a mixture of sucrose palmitate(s) and sucrose stearate(s), b) about 0.6% to 1.3% saponins, e.g., saponins derived from Quillaja saponaria, and about 1% to 2.6% lecithins e.g., de-oiled soy lecithin as secondary surfactants, c) about 4% to 8% non-polar solvent(s), e.g., olive oil, d) about 70% to 85% polar solvent(s), e.g., a combination of water and glycerin, and e) about 4% to 8% non-polar active ingredient(s), e.g., cannabidiol.
1.50. Any previous formulation, wherein the nanoemulsion concentrate comprises, by weight: a) about 5% to 7.5% sucrose fatty acid esters, e.g., a mixture of sucrose palmitate(s) and sucrose stearate(s), b) about 0.7% to 1.2% saponins, e.g., saponins derived from Quillaja saponaria, and about 1.1% to 2.5% lecithins e.g., de-oiled soy lecithin as secondary surfactants, c) about 5% to 7.5% non-polar solvent(s), e.g., olive oil, d) about 70% to 85% polar solvent(s), e.g., a combination of water and glycerin, and e) about 5% to 7.5% non-polar active ingredient(s), e.g., cannabidiol.
1.51. Any previous formulation, wherein the nanoemulsion concentrate comprises, by weight: a) about 4.5% to 6% sucrose fatty acid esters, e.g., a mixture of sucrose palmitate(s) and sucrose stearate(s), b) about 0.6% to 1% saponins, e.g., saponins derived from Quillaja saponaria, and about 1% to 1.8%% lecithins e.g., de-oiled soy lecithin as secondary surfactants, c) about 4% to 6% non-polar solvent(s), e.g., olive oil, d) about 80% to 85% polar solvent(s), e.g., a combination of water and glycerin, and e) about 4.5% to 5.5% non-polar active ingredient(s), e.g., cannabidiol.
1.52. Any previous formulation, wherein the nanoemulsion concentrate comprises, by weight: a) about 4.5% to 5.5% sucrose fatty acid esters, e.g., a mixture of sucrose palmitate(s) and sucrose stearate(s), b) about 0.7% to 0.8% saponins, e.g., saponins derived from Quillaja saponaria, and about 1.5% to 1.8%% lecithins e.g., de-oiled soy lecithin as secondary surfactants, c) about 4% to 6% non-polar solvent(s), e.g., olive oil, d) about 80% to 85% polar solvent(s), e.g., a combination of water and glycerin, and e) about 4.5% to 5.5% non-polar active ingredient(s), e.g., cannabidiol.
1.53. Any previous formulation, wherein the nanoemulsion concentrate comprises, by weight: a) about 5% to 6% sucrose fatty acid esters, e.g., a mixture of sucrose palmitate(s) and sucrose stearate(s), b) about 0.7% to 1% saponins, e.g., saponins derived from Quillaja saponaria, and about 1% to 1.3% lecithins e.g., de-oiled soy lecithin as secondary surfactants, c) about 4% to 6% non-polar solvent(s), e.g., olive oil, d) about 80% to 85% polar solvent(s), e.g., a combination of water and glycerin, and e) 4.5%-5.5% non-polar active ingredient(s), e.g., cannabidiol.
1.54. Any previous formulation, wherein the nanoemulsion concentrate comprises, by weight: a) about 7% to 8% sucrose fatty acid esters, e.g., a mixture of sucrose palmitate(s) and sucrose stearate(s), b) about 1% to 1.2% saponins, e.g., saponins derived from Quillaja saponaria, and about 2.3% to 2.7% lecithins e.g., de-oiled soy lecithin as secondary surfactants, c) about 7% to 8% non-polar solvent(s), e.g., olive oil, d) about 70% to 75% polar solvent(s), e.g., a combination of water and glycerin, and e) about 7% to 8% non-polar active ingredient(s), e.g., cannabidiol.
1.55. Any previous formulation, wherein the nanoemulsion concentrate comprises, by weight, about: a) about 5% sucrose fatty acid esters, specifically a mixture of sucrose palmitate(s) and sucrose stearate(s) with an HLB of about 16, as the primary surfactant, b) about 0.8% saponins, derived from Quillaja saponaria, and about 1.7% de-oiled soy lecithin as secondary surfactants, c) about 5% olive oil as a non-polar solvent, d) about 79% water and about 3% glycerin as the aqueous phase, and e) about 5% cannabidiol as the non-polar active ingredient.
1.56. Any previous formulation, wherein the nanoemulsion concentrate comprises, by weight, about: a) about 5% sucrose fatty acid esters, specifically a mixture of sucrose palmitate(s) and sucrose stearate(s) with an HLB of about 16, as the primary surfactant, b) about 0.8% saponins, derived from Quillaja saponaria, and about 1.7% de-oiled soy lecithin as secondary surfactants, c) about 5% olive oil as a non-polar solvent, d) about 79% water and about 3% glycerin as the aqueous phase, and e) about 5% hemp seed as the non-polar active ingredient.
1.57. Any previous formulation, wherein the nanoemulsion concentrate comprises, by weight, about: a) about 5.5% sucrose fatty acid esters, specifically a blend of sucrose palmitate(s) and sucrose stearate(s) with an HLB of about 16, as the primary surfactant, b) about 0.9% saponins, derived from Quillaja saponaria, and about 1.1% de-oiled soy lecithin as secondary surfactants, c) about 5% olive oil as a non-polar solvent, d) about 79% water and about 3% glycerin as the aqueous phase, and e) about 5% cannabidiol as the non-polar active ingredient.
1.58. Any previous formulation, wherein the nanoemulsion concentrate comprises, by weight, about: a) about 7.5% sucrose fatty acid esters, specifically a mixture of sucrose palmitate(s) and sucrose stearate(s) with an HLB of about 16, as the primary surfactant, b) about 1.1% saponins, derived from Quillaja saponaria, and about 2.5% de-oiled soy lecithin as secondary surfactants, c) about 7.5% olive oil as a non-polar solvent, d) about 71% water and about 3% glycerin as the aqueous phase, and e) about 5% cannabidiol as the non-polar active ingredient.
1.59. Any previous formulation wherein the pH is 4.5 or less.
1.60. Any previous formulation wherein the pH is 2.5 to 4.5.
1.61. Any previous formulation wherein the pH is pH 3.2 to pH 4.2.
1.62. Any previous formulation which is a beverage.
1.63. Any previous formulation which further comprises sweeteners, flavorants, and or coloring agents.
1.64. Any previous formulation which is palatable and non-toxic.
1.65. Any previous formulation which comprises an acidulant, e.g., selected from citric acid, lactic acid, malic acid, phosphoric acid and tartaric acid, for example citric acid.
1.66. Any previous formulation which comprises a preservative, e.g. potassium sorbate.
1.67. Any previous formulation wherein:
   a) the primary surfactant comprises about 5% of a blend of sucrose palmitate(s) and sucrose stearate(s) with an HLB of about 16;
   b) the secondary surfactants comprise about 0.7% saponins, derived from Quillaja saponaria, and about 1.7% de-oiled soy lecithin;
   c) the non-polar solvent comprises about 5% olive oil and about 0.5% coconut oil;
   d) the aqueous phase comprises about 78.8% water and about 3.3% glycerin; and
   e) the non-polar active ingredient is about 5% cannabidiol;
   wherein all weight percents are by weight of the formulation.
1.68. Any previous formulation wherein:
   a) the primary surfactant comprises about 7.5% of a blend of sucrose palmitate(s) and sucrose stearate(s) with an HLB of about 16;
   b) the secondary surfactants comprise about 1.1% saponins, derived from Quillaja saponaria, and about 2.5% de-oiled soy lecithin;
   c) the non-polar solvent comprises about 7.5% olive oil;
   d) the aqueous phase comprises about 68.9% water and about 5% glycerin; and
   e) the non-polar active ingredient is about 7.5% cannabidiol;
   wherein all weight percents are by weight of the formulation.
1.69. Any previous formulation wherein:
   a) the primary surfactant comprises about 5% of a blend of sucrose palmitate(s) and sucrose stearate(s) with an HLB of about 16;
   b) the secondary surfactants comprise about 0.7% saponins, derived from Quillaja saponaria, and about 1.7% de-oiled soy lecithin;
   c) the non-polar solvent comprises about 5% olive oil and about 1.3% fractionated coconut oil;
   d) the aqueous phase comprises about 76.3% water and about 5% glycerin; and
   e) the non-polar active ingredient is about 5% cannabidiol;
   wherein all weight percents are by weight of the formulation.
1.70. Any previous formulation wherein:
   a) the primary surfactant comprises about 7.5% sucrose fatty acid esters, e.g., a blend of sucrose palmitate(s) and sucrose stearate(s) with an HLB of about 16,
   b) the secondary surfactants comprise about 1.1% saponins derived from Quillaja saponaria, and about 2.5% de-oiled soy lecithin,
   c) the non-polar solvent comprises about 7.5% olive oil, and about 0.75% coconut oil, e.g., including lauric acid,
   d) the aqueous phase comprises about 70.65% water and about 5.0%,
   e) the non-polar active ingredient is about 5.0% cannabidiol;
   wherein all weight percents are by weight of the formulation.
1.71. Any previous formulation wherein:
   a) the primary surfactant is about 5% of a blend of sucrose palmitate(s) and sucrose stearate(s) with an HLB of about 16;
   b) the secondary surfactants are about 0.7% saponins, derived from Quillaja saponaria, and about 1.7% de-oiled soy lecithin;
   c) the non-polar solvent comprises about 5% olive oil and about 1.3% fractionated coconut oil;
   d) the aqueous phase comprises about 76.3% water and about 5% glycerin;
   e) the non-polar active ingredient is about 5% cannabidiol; and
   f) further comprising about 0.5% mixed tocopherols as an oil-soluble antioxidant;
   wherein all weight percents are by weight of the formulation.

In a further embodiment, the disclosure provides an oil/surfactant pre-mixture (Formulation 2), comprising:
a. a primary surfactant comprising a sucrose fatty acid ester or a mixture of sucrose fatty acid esters;
b. one or more secondary surfactants selected from among lecithins, saponins, and mixtures thereof;
c. one or more non-polar solvents; and
d. a non-polar active ingredient.

For example, the disclosure provides:
2.1. Formulation 2 wherein the sucrose fatty acid ester or a mixture of sucrose fatty acid esters is selected from the group consisting of sucrose palmitates, sucrose stearates, and mixtures thereof.
2.2. Formulation 2 or 2.1, wherein the primary surfactant is a mixture of sucrose palmitate(s) and sucrose stearate(s), optionally wherein the mixture of sucrose palmitate(s) and sucrose stearate(s) has an overall monoester content of about 75% and a diester content of about 20% and has an HLB of about 16.
2.3. Any previous formulation, wherein the sucrose fatty acid ester or mixture of sucrose fatty acid esters has an HLB of about 14 to 16.
2.4. Any previous formulation, wherein the secondary surfactant comprises liquid lecithins, de-oiled lecithins, modified lecithins, or mixtures thereof.
2.5. Any previous formulation, wherein the secondary surfactant comprises a saponin, e.g., a saponin derived from Quillaja saponaria.
2.6. Any previous formulation, wherein the secondary surfactants comprise a mixture of lecithins and saponins.
2.7. Any previous formulation, wherein the secondary surfactants comprise a mixture of a de-oiled lecithin and one or more saponins.
2.8. Any previous formulation wherein the one or more nonpolar solvents comprise triglycerides.
2.9. Any previous formulation wherein the one or more nonpolar solvents are selected from medium-chain triglycerides, long-chain triglycerides, coconut oil, fractionated coconut oil, olive oil, corn oil, sunflower oil, fish oil, and mixtures thereof
2.10. Any previous formulation, wherein the one or more nonpolar solvents comprise a long-chain triglyceride-containing non-polar solvent, e.g., olive oil.
2.11. Any previous formulation, wherein the one or more nonpolar solvents comprise a medium-chain triglyceride-containing non-polar solvent, e.g., coconut oil.
2.12. Any previous formulation, wherein the one or more nonpolar solvents comprise a combination of a long-chain triglyceride-containing non-polar solvents and a medium-chain triglyceride-containing solvents, e.g., a combination of olive oil and coconut oil.
2.13. Any previous formulation wherein the non-polar active ingredient is selected from non-polar cannabinoid compounds.
2.14. Any previous formulation wherein the non-polar active ingredient is selected from cannabidiol, tetrahydrocannabinol, hemp or cannabis plant extracts containing greater than 50% total cannabinoids, and mixtures thereof
2.15. Any previous formulation wherein the non-polar active ingredient is cannabidiol.
2.16. Any previous formulation wherein the non-polar active ingredient is tetrahydrocannabinol.
2.17. Any previous formulation wherein the non-polar active ingredient is hemp oil.
2.18. Any previous formulation wherein the non-polar active ingredient is cannabidiol, wherein the cannabidiol is substantially free of other cannabinoids, e.g., at least 99% free of other cannabinoids.
2.19. Any previous formulation wherein the non-polar active ingredient is cannabidiol, wherein the cannabidiol is substantially free of other cannabinoids, e.g., at least 99% free of other cannabinoids, and is isolated from hemp.
2.20. Any previous formulation wherein the total surfactants in the nanoemulsion have an overall HLB of about 13 or higher.
2.21. Any previous formulation further comprising an oil-soluble antioxidant.
2.22. Any previous formulation further comprising mixed tocopherols as an oil-soluble antioxidant.
2.23. Any previous formulation that, upon addition to an aqueous medium, forms a liquid nanoemulsion.
2.24. Any previous formulation formed by drying or lyophilizing a formulation of any of Formulation 1, et seq.
2.25. Any previous formulation wherein the total amount of sucrose fatty acid esters is between about 25% and about 45%, e.g., from 25% to 40%, from 25% to 35%, from 27% to from 33%, or from 25% to 30%, by weight of the pre-mixture.
2.26. Any previous formulation wherein the total amount of secondary surfactants is between about 5% and about 25%, e.g., from 10% to 20%, or from 10% to 15%, by weight of the pre-mixture.
2.27. Any previous formulation, wherein the lecithins are present in an amount of from 4% to 15%, e.g., from 5% to 12%, or from 6% to 10% by weight of the pre-mixture and the saponins are present in an amount of from 2% to 6%, e.g., from 3% to 5.5%, or from 4% to 5% by weight of the pre-mixture.
2.28. Any previous formulation wherein the total amount of the one or more non-polar solvents in an amount between about 25% and about 45%, e.g., from 25% to 40%, from 25% to 35%, from 27% to from 33%, or from 25% to 30%, by weight of the pre-mixture.
2.29. Any previous formulation wherein the total amount of the non-polar active ingredient in an amount between about 25% and about 45%, e.g., from 25% to 40%, from 25% to 35%, from 27% to from 33%, or from 25% to 30%, by weight of the pre-mixture.
2.30. Any previous formulations further comprising one or more non-water polar solvents in a total amount between about 1% and about 25%, by weight of the pre-mixture.
2.31. Any previous formulations further comprising one or more non-water polar solvents in a total amount between about 1% and about 25%, by weight of the pre-mixture, wherein the non-water polar solvents are selected from glycerin, propylene glycol, and mixtures thereof.
2.32. Any previous formulation comprising
   a) a mixture of sucrose fatty acid esters primarily composed of sucrose palmitate;
   b) a de-oiled soy lecithin;
   c) a saponin, e.g., saponin derived from Quillaja saponaria;
   c) olive oil as a source of long-chain triglycerides; and
   d) glycerol as a non-water polar solvent.
2.33. Any previous formulation comprising
   a) a mixture of sucrose fatty acid esters primarily composed of sucrose palmitate;
   b) a de-oiled soy lecithin;
   c) olive oil as a source of long-chain triglycerides; and
   d) glycerol as a non-water polar solvent.

In another embodiment, the disclosure provides a method of providing an oil-based additive in a beverage, comprising: adding the liquid nanoemulsion concentrate of any of Formulation 1, et seq. or adding the oil/surfactant pre-mixture of any of Formulation 2, et seq., to an aqueous beverage medium in an amount to provide an effective amount of the non-polar active ingredient for supplementation of the diet or achieving a desired pharmacological effect.

In another embodiment, the disclosure provides a method for preparing a powder, comprising spray drying or freeze drying the liquid nanoemulsion concentrate of any of Formulation 1, et seq., e.g., to provide any of Formulation 2, et seq.

In another embodiment, the disclosure provides a method of producing a nanoemulsion concentrate of any of Formulation 1, et seq., comprising:
a. combining an oil/surfactant pre-mixture of any of Formulation 2, et seq., with one or more non-polar active ingredients; then
b. combining the resulting mixture with an aqueous pre-mixture that contains, by weight, greater than 50% water; then
c. processing the resulting mixture with an ultrasonic homogenizer or high pressure homogenizer to produce a nanoemulsion concentrate.

In another embodiment, the disclosure provides a method of producing a nanoemulsion concentrate of any of Formulation 1, et seq., comprising:
a. combining the oil/surfactant pre-mixture according to any of Formulation 2, et seq., with one or more non-polar active ingredients; then
b. combining the resulting mixture with an aqueous component that contains both water and a mixture of saponins derived from Quillaja saponaria; then
c. processing the resulting mixture with an ultrasonic homogenizer or high pressure homogenizer to produce a nanoemulsion concentrate.

In another embodiment, the disclosure provides a method of treating a condition selected from pain, anxiety, nausea, dysphoria, insomnia, neuroinflammation, spasms, epilepsy, inflammation, Alzheimer's Disease, Amyotrophic Lateral Sclerosis (ALS), chronic pain, Diabetes Mellitus, dystonia, epilepsy, fibromyalgia, gastrointestinal disorders, gliomas/cancer, Hepatitis C, Human Immunodeficiency Virus (HIV), Huntington Disease, Hypertension, Incontinence, Methicillin-resistant Staphyloccus aureus (MRSA), Multiple Sclerosis, osteoporosis, pruritus, rheumatoid arthritis, sleep apnea, Parkinson's disease, chronic inflammation, chronic pain, cancer, nausea, vomiting, obesity, epilepsy, glaucoma, asthma, mood disorders, and Tourette Syndrome, comprising administering an effective amount of a pharmaceutical composition according to any of Formulation 1, et seq., or Formulation 2, et seq., to a patient in need thereof, wherein the active ingredient in the pharmaceutical composition according to any of Formulation 1, et seq., or Formulation 2, et seq., is a cannabinoid, e.g., selected from CBD, THC and combinations thereof.

For example, the disclosure provides a method of treating a condition selected from pain, anxiety, nausea, insomnia, and dysphoria comprising administering an effective amount of a pharmaceutical composition according to any of Formulation 1, et seq., or Formulation 2, et seq., to a patient in need thereof, wherein the active ingredient in the pharmaceutical composition according to any of Formulation 1, et seq., or Formulation 2, et seq., is a cannabinoid, e.g., selected from CBD, THC and combinations thereof, e.g., wherein the active ingredient is CBD.

In another embodiment, the disclosure provides a beverage product comprising any of Formulation 1, et seq. or Formulation 2, et seq., e.g., prepared by adding any of Formulation 1, et seq. or Formulation 2, et seq., to a beverage. For example, the disclosure provides a beverage comprising any of Formulation 1, et seq. or Formulation 2, et seq., wherein the pH is 4.5 or less, e.g., pH 2.5 to pH 4.5, e.g., pH 3.2 to pH 4.2. In certain embodiments, the beverage product comprises sweetener(s), flavoring(s), and/or colorant(s). In certain embodiments, beverage product further comprises an acidulant, e.g., citric acid. In certain embodiments, the beverage product further comprises a preservative, e.g., potassium sorbate.

In another embodiment, the disclosure provides a capsule, e.g., a soft gel capsule, containing any of Formulation 1, et seq. or Formulation 2, et seq.

Unless stated otherwise, all percentages of composition components given in this specification are by weight based on a total composition or formulation weight of 100%.

The compositions and formulations as provided herein are described and claimed with reference to their ingredients, as is usual in the art. As would be evident to one skilled in the art, the ingredients may in some instances react with one another, so that the true composition of the final formulation may not correspond exactly to the ingredients listed. Thus, it should be understood that the invention extends to the product of the combination of the listed ingredients.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

### EXAMPLES

Ten different cannabinoid-containing oil-in-water nanoemulsion formulations (emulsions 1-10) were prepared as indicated in Table 1.

TABLE 1

| Emulsion Number | | 2 | | | |
|---|---|---|---|---|---|
| Active Ingredient | 1 | Hemp | 3 | 4 | 5 |
| Identify | CBD | Extract | CBD | CBD | CBD |
| Active Ingredient (%) | 5 | 5 | 5 | 7.5 | 10 |
| Sucrose Esters (%) | 5 | 5 | 5.5 | 7.5 | 10 |
| Lecithin (%) | 1.65 | 1.65 | 1.1 | 2.5 | 3.3 |
| Saponin (%) | 0.76 | 0.76 | 0.85 | 1.14 | 1.52 |
| Olive Oil (%) | 5 | 5 | 5 | 7.5 | 10 |
| Glycerol (%) | 3.3 | 3.3 | 3.3 | 2.95 | 2.6 |
| Water (%) | 79.29 | 79.29 | 79.25 | 70.91 | 62.58 |
| Primary Surfactant (%) | 5 | 5 | 5.5 | 7.5 | 10 |
| Secondary Surfactant (%) | 2.41 | 2.41 | 1.95 | 3.64 | 4.82 |
| Non Polar Solvents (%) | 5 | 5 | 5 | 7.5 | 10 |
| Polar Solvent (%) | 82.59 | 82.59 | 82.6 | 73.9 | 65.2 |
| Overall HLB | 13.74 | 13.74 | 14.39 | 13.72 | 13.74 |
| Surfactant:Oil Ratio | 0.741 | 0.741 | 0.745 | 0.743 | 0.741 |
| Emulsion Number | | | | | |
| Active Ingredient | 6 | 7 | 8 | 9 | 10 |
| Identify | CBD | CBD | CBD | CBD | CBD |
| Active Ingredient (%) | 2.5 | 5 | 5 | 5 | 5 |
| Sucrose Esters (%) | 2.5 | 7.41 | 5.55 | 5 | 6.45 |
| Lecithin (%) | 0.83 | 0 | 1.88 | 0 | 0 |
| Saponin (%) | 0.38 | 0 | 0 | 2.43 | 0.98 |
| Olive Oil (%) | 2.5 | 5 | 5 | 5 | 5 |
| Glycerol (%) | 3.65 | 3.3 | 3.3 | 3.3 | 3.3 |
| Water (%) | 87.65 | 79.29 | 79.28 | 79.28 | 79.28 |
| Primary Surfactant (%) | 2.5 | 7.41 | 5.55 | 5 | 6.45 |
| Secondary Surfactant (%) | 1.21 | 0 | 1.88 | 2.43 | 0.98 |
| Non Polar Solvents (%) | 2.5 | 5 | 5 | 5 | 5 |
| Polar Solvent (%) | 91.3 | 82.6 | 82.6 | 82.6 | 82.6 |
| Overall HLB | 13.74 | 16 | 13.73 | 15.18 | 15.67 |
| Surfactant:Oil Ratio | 0.741 | 0.741 | 0.743 | 0.743 | 0.743 |

The primary surfactant used in all ten formulations is an approximate 80:20 blend of sucrose palmitate and stearate which has an overall monoester content of approximately 75% and a diester content of approximately 20% (HLB 16). Secondary surfactants used between the ten formulations are de-oiled soy lecithin composed of >97% acetone insolubles (HLB 7) and/or saponin derived from Quillaja saponaria (HLB 13.5, sourced from a commercially-available water-based solution which contains approx. 14% active saponins by weight). The nonpolar solvent used in all ten formulations is an extra light olive oil which is low in taste. The two polar solvents used in all ten formulations are glycerol and water. Active ingredients used in the ten formulations are 99%+ pure CBD (emulsions 1 and 3-10) or a full-spectrum hemp extract which contains approximately 67% CBD, 3% THC, 2% terpenes, various minor cannabinoids, and other plant oils (emulsion 2). In order to allow for the most straightforward comparison, the surfactant to oil ratio (SOR) was held roughly constant during the production of all 10 emulsions (SOR range: 0.74-0.75). Wherever possible, the overall HLB of the surfactant systems has also been held constant (HLB 13.7 in 6 of the 10 emulsions). All formulations were processed in the same manner with a Misonix S-4000 ultrasonic homogenizer equipped with an ultrasonic transducer and 0.75-inch horn capable of generating an amplitude of 120 microns at maximum power. All 60 milliliter emulsion samples were processed at an amplitude of 82 microns (peak-to-peak) for 9 minutes (approximately 60,000 Joules of power, as indicated by the S-4000 unit) at a temperature of 65° C. The resulting nanoemulsions were then filtered through a 0.22 micron polyether sulfone membrane filter to remove any residual metal particles shed from the titanium horn. All emulsions were then tested for particle size and many were also assessed for stability at acidic pH, taste and optical properties in model beverages and other liquid dosage forms. The results are shown in Table 2.

TABLE 2

| | | | | | |
|---|---|---|---|---|---|
| Emulsion Number | 1 | 2 | 3 | 4 | 5 |

Particle Size Data

| | | | | | |
|---|---|---|---|---|---|
| Volume-Weighted | 32.6 | 30.4 | 28 | 31.7 | 48.8 |
| Average Size (nm) | | | | | |
| Dv90 (nm) | 56.3 | 50.4 | 44.4 | 53.8 | 107.1 |
| Dispersed Phase Volume | 99.9 | 99.8 | 100 | 99.7 | 88.2 |
| Smaller Than 100 nm (%) | | | | | |

Water Beverage Taste and Clarity Data

| | | | | | |
|---|---|---|---|---|---|
| Visual Clarity in Beverage | Clear | Clear | Clear | Clear | Slightly |
| (20 mg Active/16 oz Pure | | | | | Turbid |
| Water) | | | | | |
| Taste in Beverage | Near | Slight | Near | Near | Near |
| (10 mg Active/16.9 oz | Tasteless | Cannabis- | Tasteless | Tasteless | Tasteless |
| Water) | | like Flavor | | | |
| Taste in Beverage | Slightly | Cannabis- | Slightly | Slightly | Slightly |
| (20 mg Active/16.9 oz | Bitter | like Flavor | Bitter | Bitter | Bitter |
| Water) | | | | | |

Shot and Concentrated Supplement Taste Data

| | | | | | |
|---|---|---|---|---|---|
| Taste in Shot | No | No | No | Not Tested | Not |
| (15 mg active/60 ml Shot) | Unpleasant | Unpleasant | Unpleasant | | Tested |
| | Taste | Taste | Taste | | |
| Taste in Concentrated | Mild Bitter, | Mild | Mild Bitter, | Not Tested | Not |
| Supplement | Mild Throat | Throat Burn | Mild Throat | | Tested |
| (5 mg Active/0.5 ml Oral | Burn | | Burn | | |
| Dose) | | | | | |

Stability at pH 4.0 Data

| Pellet Volume | 5 uL | <5 uL | 15 uL | 5 uL | Not |
|---|---|---|---|---|---|
| (25 mg/ml Active, pH 4.0 | | | | | Tested |
| for 48 hrs, 2 min Spin) | | | | | |
| Supernatant Color | Light yellow | Amber | Light yellow | Light yellow | Not |
| (25 mg/ml Active, pH 4.0 | | | | | Tested |
| for 48 hrs, 2 min Spin) | | | | | |
| Supernatant Clarity | Transparent | Transparent | Transparent | Transparent | Not |
| (25 mg/ml Active, pH 4.0 | | | | | Tested |
| for 48 hrs, 2 min Spin) | | | | | |
| Emulsion Number | 6 | 7 | 8 | 9 | 10 |

Particle Size Data

| | | | | | |
|---|---|---|---|---|---|
| Volume-Weighted | 51.5 | 52 | 46 | 32.6 | 25.1 |
| Average Size (nm) | | | | | |
| Dv90 (nm) | 114.4 | 67.2 | 95.8 | 46.8 | 38.2 |
| Dispersed Phase Volume | 83.1 | 94.6 | 91.5 | 99 | 100 |
| Smaller Than 100 nm (%) | | | | | |

Water Beverage Taste and Clarity Data

| | | | | | |
|---|---|---|---|---|---|
| Visual Clarity in Beverage | Slightly | Nearly | Slightly | Clear | Clear |
| (20 mg Active/16 oz Pure | Turbid | Clear | Turbid | | |
| Water) | | | | | |
| Taste in Beverage | Near | Near | Near | Slightly | Near |
| (10 mg Active/16.9 oz | Tasteless | Tasteless | Tasteless | Bitter/ | Tasteless |
| Water) | | | | Slightly | |
| | | | | Astringent | |
| Taste in Beverage | Slightly | Slightly | Slightly | Bitter/ | Slightly |
| (20 mg Active/16.9 oz | Bitter | Bitter | Bitter | Slightly | Bitter |
| Water) | | | | Astringent | |

Shot and Concentrated Supplement Taste Data

| | | | | | |
|---|---|---|---|---|---|
| Taste in Shot | Not | Not | No | Mild Bitter, | Not |
| (15 mg active/ 60 ml Shot) | Tested | Tested | Unpleasant | Mild | Tested |
| | | | Taste | Astringent, | |
| | | | | Mild Throat | |
| | | | | Burn | |
| Taste in Concentrated | Not | Not | Mild Bitter, | Moderate | Not |
| Supplement | Tested | Tested | Mild Throat | Bitter, | Tested |
| (5 mg Active/0.5 ml Oral | | | Burn | Moderate | |
| Dose) | | | | Astringent, | |
| | | | | Strong | |
| | | | | Throat Burn | |

Stability at pH 4.0 Data

| | | | | | |
|---|---|---|---|---|---|
| Pellet Volume | Not | 300 uL | 25 uL | 10 uL | 20 uL |
| (25 mg/ml Active, pH 4.0 | Tested | | | | |
| for 48 hrs, 2 min Spin) | | | | | |
| Supernatant Color | Not | Colorless | Cream | Amber | Cream |
| (25 mg/ml Active, pH 4.0 | Tested | | | | |
| for 48 hrs, 2 min Spin) | | | | | |
| Supernatant Clarity | Not | Clear | Slightly | Translucent | Opaque |
| (25 mg/ml Active, pH 4.0 | Tested | | Translucent | | |
| for 48 hrs, 2 min Spin) | | | | | |

Particle size data was acquired via Dynamic Light Scattering (DLS), also known as photon correlation spectroscopy, which was performed on a Beckman Coulter N4 Plus unit. Four sets of particle size data were collected for each sample. This data was then processed via CONTIN algorithm to yield four volume-weighted particle size distributions for each sample. These volume-weighted size distributions were then averaged for each sample and the following values were calculated for each of the ten emulsions: volume-weighted average particle size, the 90^{th} percentile volume-weighted particle size (Dv90), and the volume-percentage of dispersed-phase material contained within particles smaller than 100 nm (u100 nm). These metrics correlate to theoretical bioavailability (average particle size), emulsion stability (average particle size and Dv90 indicate distribution width and resistance to ostwald ripening) and the optical clarity of the emulsions (u100 nm indicates the percentage of particles too small to scatter visible light). These data are displayed in Table 2.

Nanoemulsions 1, 2, 3, 4, 7, 8, 9, and 10 were then tested for stability at acidic pH. For this test, a pH of 4.0 was chosen because many preserved beverages and supplements require a pH of less than 4.2 for adequate preservation. A portion of each sample was diluted to achieve an active ingredient concentration of 25 mg/ml and simultaneously acidified to pH 4.0+/−0.05 via the addition of a citric-acid/potassium sorbate solution. All acidified samples were stored at room temperature for 48 hours prior to observation. After 48 hours, samples were inverted quickly 10 times to ensure homogeneity and 1.25 ml of each acidified emulsion sample was loaded into a 1.7 ml microfuge tube. To assess emulsion stability, each 1.25 ml sample was centrifuged for 2 minutes in a small benchtop centrifuge. The following stability-indicating parameters were observed/assessed and are shown in Table 2: estimated pellet volume, supernatant color, and supernatant clarity. Pellet formation upon centrifugation, and lack of supernatant clarity generally indicate nanoparticle aggregation and/or growth. Supernatant color can be useful in assessing whether the supernatant still contains any appreciable quantity of oil-based nanoparticles (in this case, clear and colorless supernatant in conjunction with large pellet volume would indicate near total emulsion destabilization).

To evaluate the optical properties and taste of the emulsions in a dilute flavorless beverage, emulsions 1-10 were each added to two 16.9 oz clear PET bottles of reverse-osmosis purified water with no added minerals (Aquafina brand) to achieve active ingredient concentrations of 10 mg and 20 mg per bottle for each of the 10 emulsions. In a well-lit room, the optical clarity of the water bottles containing 20 mg of active ingredient was observed against a water bottle which did not contain any emulsion. A grade was reported for the clarity of each emulsion-containing bottle from the following options: clear, nearly clear, slightly turbid, turbid. These Data are reported in Table 2. In order to assess taste, a group of three taste-testers was assembled and instructed to sample 30 ml of each of the 20 emulsion-containing beverages and agree on one or more descriptors for each. In order to ensure a fresh pallet, a minimum of 10 minutes between tastings was enforced and tasting was spread over the course of two days. The allowable descriptors for reporting results were as follows: tasteless, near tasteless, slightly bitter, bitter, slightly astringent, astringent, slight cannabis-like flavor, cannabis-like flavor. Results are reported in Table 2.

To evaluate the taste of the emulsions in a model shot and a concentrated multi-dose supplement product, another set of taste tests was conducted with emulsions 1-3 and 8-9. Model 2.0 oz shots were produced to mimic a typical formula of a water-based hot-fill compliant supplement shot: pH 4.1 (adjusted with citric acid), a potassium sorbate concentration of 0.1%, monk fruit extract and glycerol as sweeteners, gum Arabic as a flavor enhancer, and artificial pineapple flavor. A sufficient amount of emulsions 1-3 and 8-9 was added to each sample of this shot base to achieve a 15 mg dose of active ingredient per 2.0 oz of shot base liquid. Model concentrated multi-dose supplements were produced to mimic the typical formula of a water-based hot-fill compliant multi-dose supplement: pH 4.1 (adjusted with citric acid), a potassium sorbate concentration of 0.1%, monk fruit extract, glycerol and honey as sweeteners, gum Arabic as a flavor enhancer, a commercially available mushroom-derived flavor enhancer (bitter-blocker), and artificial pineapple flavor. A sufficient amount of emulsions 1-3 and 8-9 was added to each sample of this supplement base to achieve a 10 mg dose of active ingredient per 1.0 ml of supplement base liquid. The taste tests for the model shot and model concentrated multi-dose supplement product were conducted in a similar manner to the taste testing described previously. For this set of taste tests, subjects consumed 0.5 oz of each model shot, and 0.5 ml of each model concentrated multi-dose supplement over two days. Allowed descriptors were as follows: no unpleasant taste, mild bitter, moderate bitter, strong bitter, mild throat burn, moderate throat burn, strong throat burn, mild astringent, moderate astringent, strong astringent. Results are reported in Table 2.

In order to allow for the most straightforward comparison, the surfactant to oil ratio was held roughly constant during the production of all 10 emulsions presented in Table 1. Wherever possible, the overall HLB of the surfactant systems was also been held constant. The data presented in Table 2 illustrates that a combination of surfactants is superior to sucrose esters alone in terms of average particle size achieved, size distribution and emulsion stability at low pH. Emulsion 7, which contains only sucrose esters with no secondary surfactant, has an average particle size of 52.0 nm, which is substantially larger than emulsions 1-4 (28.0-32.6 nm) and emulsions 9-10 (25.1-32.6 nm), which may lead to reduced bioavailability of the active ingredient in emulsion 7, compared to emulsions 1-4 or 9-10. Furthermore, the particle size distribution of emulsion 7 (Dv90=67.2 nm, u100 nm=94.6%) is inferior to that of emulsions 1-4 (Dv90=44.4-56.3, u100 nm=99.7-100.0%) and 9-10 (Dv90=38.2-46.8 nm, u100 nm=99.0-100.0%), leading to less optical clarity in both concentrated and dilute finished products. Emulsion 7 is also unstable at a pH of 4.0, showing rapid particle aggregation and near total phase separation, whereas emulsions 1-4 are relatively stable under the same conditions. Therefore, sucrose esters alone are not as suitable for the nanoemulsification of cannabinoids as several other combinations of surfactants.

Emulsions 9 and 10 contain only two surfactants, sucrose esters and saponin. While the initial particle size achieved with these formulations is close to that of emulsions 1-4, which contain three surfactants, emulsions 9 and 10 are substantially less stable at pH 4.0 than several of the three-surfactant emulsions presented. This lack of stability is evidenced by the opaque, cream-colored appearance of emulsion 10 after 48 hours at pH 4.0 (emulsion 10 was highly transparent at the time of production) and the 20 uL pellet volume upon brief centrifugation, while emulsions 1-4 remained transparent when subjected to the same conditions and emulsions 1, 2 and 4 had pellet volumes of 5 uL or less. Emulsion 9, though more stable than emulsion 10, still has diminished stability when compared to emulsions 1-4 as evidenced by emulsion 9 turning from transparent to translucent under acidic conditions and the 10 uL pellet volume. Furthermore, emulsion 9 has the worst taste profile among the 10 emulsions certainly due to the fact that it contains much more saponin than the other emulsions listed in Table 1. Saponin is bitter, astringent, and contributes to throat burn. Therefore, formulations relying only on saponin to reduce the particle size of sucrose ester-based cannabinoid nanoemulsions either lack stability or a palatable taste profile, or both.

Emulsion 8 also contains only two surfactants, sucrose esters and lecithin. Emulsion 8 has the same SOR as emulsions 1-4 and the same HLB as emulsions 1, 2, and 4. However, emulsion 8 performs substantially worse in terms of particle size and slightly worse in terms of stability when compared to emulsions 1-4. Emulsion 8 has average particle size of 46 nm, substantially greater than emulsions 1-4 (28.0-32.6 nm) and a Dv90 and u100 nm of 95.8 nm and 91.5%, far worse than emulsions 1-4 (Dv90=44.4-56.3 nm, u100 nm=99.7-100.0%). With brief centrifugation, emulsion 8 produces a pellet of 25 uL after 48 hours at pH 4.0 and suffers a loss of clarity, indicating particle aggregation and/or growth.

It can therefore be concluded that the combination of sucrose esters, specifically sucrose palmitate and stearate with a high monoester content, with both lecithin and saponin leads to the most well-rounded cannabinoid nanoemulsion formulation. This optimized formulation has a very small volume-weighted average particle size of approximately 30 nm, thus enhancing absorption through the body's mucous membranes and the digestive tract, leading to very high bioavailability of the active ingredient. This optimized formulation provides near perfect optical transparency in an otherwise transparent beverage or supplement, thus enhancing perceived product quality. This optimized formulation is relatively stable at pH 4.0, which makes it a suitable candidate for inclusion in many acidified preserved water-based beverage and supplement products. Because this optimized formulation is primarily composed of two surfactants which impart little taste into the finished product (sucrose esters and lecithin) and contains a relatively small amount of saponins, it still maintains a reasonable taste profile in beverages and supplements.

1. A liquid nanoemulsion concentrate, comprising:
a. a primary surfactant comprising a sucrose fatty acid ester or a mixture of sucrose fatty acid esters, wherein the total amount of sucrose fatty acid esters is between about 1% and about 15%, by weight of the concentrate;
b. one or more secondary surfactants selected from lecithins, saponins, and mixtures thereof, wherein the total amount of secondary surfactants is between about 0.3% and about 10%, by weight of the concentrate;
c. one or more non-polar solvents, in a total amount between about 1% and about 18%, by weight of the concentrate;
d. a non-polar active ingredient in a total amount between about 1% and about 15%, by weight of the concentrate; and
e. an aqueous continuous phase consisting of one or more polar solvents in a total amount between about 45% and about 96%, by weight of the concentrate.

2. The liquid nanoemulsion concentrate according to claim 1, wherein the sucrose fatty acid ester or a mixture of sucrose fatty acid esters is selected from the group consisting of sucrose palmitates, sucrose stearates, and mixtures thereof.

3. The liquid nanoemulsion concentrate according to claim 1, wherein the liquid nanoemulsion concentrate comprises between about 4.5% and about 12.5%, by weight of the concentrate, of sucrose fatty acid ester or mixture thereof.

4. The liquid nanoemulsion concentrate according to claim 1, wherein the sucrose fatty acid ester or mixture of sucrose fatty acid esters has an HLB of about 14 to 16.

5. The liquid nanoemulsion concentrate according to claim 1, wherein the one or more secondary surfactants are selected from liquid lecithins, de-oiled lecithins, modified lecithins, saponins, e.g., saponins derived from Quillaja saponaria, and mixtures thereof.

6. The liquid nanoemulsion concentrate according to claim 1, wherein the one or more secondary surfactants comprise lecithins in an amount of from 1% to 3% and saponins in an amount of from 0.5% to 1.5% by weight of the concentrate.

7. The liquid nanoemulsion concentrate according to claim 1, wherein the one or more nonpolar solvents comprise triglycerides.

8. The liquid nanoemulsion concentrate according to claim 1, wherein the one or more nonpolar solvents are selected from medium-chain triglycerides, long-chain triglycerides, coconut oil, fractionated coconut oil, olive oil, corn oil, sunflower oil, fish oil, and mixtures thereof.

9. The liquid nanoemulsion concentrate according to claim 1, wherein the non-polar active ingredient is selected from cannabidiol, tetrahydrocannabinol, hemp or cannabis plant extracts containing greater than 50% total cannabinoids, and mixtures thereof.

10. The liquid nanoemulsion concentrate according to claim 1, wherein the polar solvent is selected from water, glycerin, propylene glycol, and mixtures thereof.

11. The liquid nanoemulsion concentrate according to claim 1, wherein the average oil-phase particle diameter is less than 100 nm or less than 50 nm.

12. The liquid nanoemulsion concentrate according to claim 1 further comprising mixed tocopherols as an oil-soluble antioxidant.

13. The liquid nanoemulsion concentrate according to claim 1, wherein the weight percentage of the surfactants does not exceed that of the non-polar components.

14. The liquid nanoemulsion concentrate according to claim 1, wherein the liquid nanoemulsion concentrate comprises, by weight of the concentrate,
a. about 5% to 7.5% sucrose fatty acid esters, about 2.4% to 3.6% secondary surfactants, about 5% to 7.5% non-polar solvents, about 5% to 7.5% non-polar active ingredient, and about 73.9% to 82.6% polar solvents; or
b. about 5% to 7.5% sucrose fatty acid esters, about 2.4% to 3.6% secondary surfactants, about 6.3% to 9.5% non-polar solvents, about 5% to 7.5% non-polar active ingredient, and about 71.9% to 81.3% polar solvents.

15. The liquid nanoemulsion concentrate according to claim 1, wherein the liquid nanoemulsion concentrate comprises, by weight of the concentrate,
a. about 5% to 7.5% sucrose fatty acid esters, e.g., a mixture of sucrose palmitates and sucrose stearates,
b. about 0.7% to 1.2% saponins, e.g., saponins derived from Quillaja saponaria, and about 1% to 2.5% lecithins e.g., de-oiled soy lecithin,
c. about 5% to 7.5% non-polar solvent(s), e.g., olive oil,
d. about 70% to 85% polar solvent(s), e.g., a combination of water and glycerin, and
e. about 5% to 7.5% non-polar active ingredient(s), e.g., cannabidiol.

16. An oil/surfactant pre-mixture, comprising:
a. a primary surfactant comprising a sucrose fatty acid ester or a mixture of sucrose fatty acid esters;
b. one or more secondary surfactants selected from lecithins, saponins, and mixtures thereof;
c. one or more non-polar solvents; and
d. a non-polar active ingredient.

17. A method of providing an oil-based additive in a beverage, comprising adding a composition according to claim 1 to an aqueous beverage medium in an amount to provide an effective amount of the non-polar active ingredient for supplementation of the diet or achieving a desired pharmacological effect.

18. A method of producing a nanoemulsion concentrate of claim 1, comprising:
a. combining an oil/surfactant pre-mixture comprising i) a primary surfactant comprising a sucrose fatty acid ester or a mixture of sucrose fatty acid esters, ii) one or more secondary surfactants selected from lecithins, saponins, and mixtures thereof, iii) one or more non-polar solvents, and iv) a non-polar active ingredient, with one or more non-polar active ingredients; then
b. combining the resulting mixture with an aqueous pre-mixture that contains, by weight, greater than 50% water; then
c. processing the resulting mixture with an ultrasonic homogenizer or high pressure homogenizer to produce a nanoemulsion concentrate.

19. A method of treating a condition selected from pain, anxiety, nausea, insomnia, and dysphoria comprising administering an effective amount of a composition according to claim 1 to a patient in need thereof, wherein the active ingredient in the composition is selected from CBD, THC and combinations thereof.

20. A beverage product to which has been added a composition according to claim 1.
